(19) United States
(12) Patent Application Publication
(10) Pub. No.: US 2026/0112459 A1
(43) Pub. Date: Apr. 23, 2026

(54) NEURAL OPTIMIZATION PLATFORM FOR POLYMER DISCOVERY

(71) Applicant: SHUBHAM CHANDRA, KATY, TX (US)

(72) Inventor: SHUBHAM CHANDRA, KATY, TX (US)

(21) Appl. No.: 18/918,565

(22) Filed: Oct. 17, 2024

(51) Int. Cl. G16C 20/30; G16C 20/70; G16C 20/90

(52) U.S. Cl. CPC G16C 20/30; G16C 20/70; G16C 20/90

(57) ABSTRACT

This platform integrates machine learning models with traditional mathematical optimization to efficiently solve complex, multi-objective problems in polymer discovery. Machine learning models, such as neural networks, generate initial polymer structures by modeling complex, non-linear relationships between various molecular properties. These initial structures are then refined through mathematical optimization techniques to ensure they meet specific constraints and performance criteria related to the desired molecular properties. This hybrid approach accelerates polymer development across various applications, including drug delivery, sustainable materials, and advanced technologies, offering tailored solutions that optimize performance, environmental sustainability, and efficiency.

### BACKGROUND

### Technical Field

This section introduces a hybrid approach combining the strengths of neural networks and traditional mathematical programming to solve complex multi-objective optimization problems. This approach utilizes neural networks to model complex, non-linear relationships and generate potential solutions, which are then refined through traditional optimization techniques to ensure adherence to predefined mathematical models and constraints This invention was developed independently, without the support of any federal or government-sponsored research or funding.

### Technical Background of the Invention

Polymer discovery is rapidly advancing through the integration of traditional experimental methods and modern computational techniques. Key advancements in this field can be categorized into several areas. High-Throughput Experimentation (HTE) is a method that utilizes automated synthesis and rapid screening to create and evaluate numerous polymer formulations simultaneously, significantly accelerating the discovery process. HTE allows for quick exploration of chemical space and efficient identification of promising polymers, though it requires substantial resources and involves complex data interpretation.

In recent years, polymer discovery has been revolutionized by computational chemistry, machine learning, and data-driven approaches, such as those enabled by the ChEMBL database. Computational chemistry and molecular modeling techniques like Molecular Dynamics (MD), Density Functional Theory (DFT), and Monte Carlo simulations predict polymer properties before synthesis, reducing the need for extensive experimental trials. However, these methods demand high computational resources and their accuracy depends on the assumptions built into the models.

The rise of machine learning and AI-driven approaches has further accelerated the polymer discovery process. Techniques such as supervised learning and generative models are employed to predict polymer properties and create novel structures based on existing data. While these methods reduce the need for exhaustive experimentation, they rely on large datasets and can present challenges in interpretability.

Combinatorial chemistry systematically generates a wide range of polymer structures by varying monomers and additives. This method is often paired with HTE for rapid testing, though large-scale synthesis can be time-consuming and costly. Additionally, rheological and mechanical analysis measures polymer properties such as strength, viscosity, and stiffness post-synthesis, providing essential data for optimizing polymer performance in real-world applications, albeit through labor-intensive processes.

Advanced quantum chemistry techniques are also used to explore electronic structures and polymer reactivity at the molecular level. These methods are critical for designing polymers with specific properties, such as conductivity or magnetism, but are computationally expensive and subject to limitations in approximation techniques.

The growing focus on sustainability has led to the discovery of plant-based biopolymers, developing biodegradable polymers from natural sources through methods like bioprospecting, genetic engineering, and enzyme-catalyzed polymerization. Although this environmentally friendly approach reduces reliance on synthetic plastics, challenges remain in scaling up production.

Polymer informatics integrates data science and materials science to predict new polymer properties using advanced algorithms and large datasets. The success of this approach heavily depends on comprehensive datasets and interdisciplinary expertise.

In this context, ChEMBL plays a crucial role by providing a large, curated database of bioactivity data on drug-like small molecules, managed by the European Bioinformatics Institute (EBI). Widely used in cheminformatics and bioinformatics research, ChEMBL offers detailed information on molecular properties, biological targets, bioactivity assays, drug metabolism, and toxicity. The ChEMBL database is a valuable resource for modeling molecular interactions, optimizing molecular structures, and predicting drug efficacy. By leveraging machine learning and computational approaches, researchers can use ChEMBL to explore chemical space and develop polymers with tailored properties. This combination of computational modeling, AI-driven discovery, and ChEMBL data allows for faster, more precise predictions in polymer discovery, advancing both experimental and computational fields.

At the core of these computational techniques are molecular fingerprints, which are compact, digital representations of chemical structures. They are used in cheminformatics to capture the presence or absence of specific molecular features, enabling algorithms to compare and analyze molecules efficiently. Fingerprints encode structural information in a binary format, where each bit typically represents the presence (1) or absence (0) of a particular molecular feature, such as a functional group or specific atom arrangement.

The ChEMBL database and molecular fingerprints share a close relationship in the fields of cheminformatics and drug discovery. ChEMBL serves as a key source of chemical and bioactivity data that can be used to generate molecular fingerprints for computational analysis. Molecular fingerprints are commonly used in various applications, including drug discovery, virtual screening, and similarity searches, where the goal is to predict the properties, activities, or interactions of molecules based on their structure.

There are two main types of molecular fingerprints:

### 1. Substructure Fingerprints:

Best for Small Molecules: These fingerprints excel at representing small molecules, commonly used in drug discovery.
How They Work: Substructure fingerprints focus on encoding specific molecular substructures, like functional groups, rings, or atomic arrangements, detecting whether a molecule contains certain key features associated with biological activity.
Applications: Widely used in pharmaceutical research, substructure fingerprints help identify molecules with important pharmacophores or functional groups crucial for drug development.

### 2. Atom-Pair Fingerprints:

Optimized for Large Molecules: Atom-pair fingerprints are suited for larger, more complex molecules such as peptides or proteins.
How They Work: These fingerprints encode the spatial distances and relationships between atom pairs, capturing more complex structural information, including how different parts of a molecule relate in 3D space.
Applications: Atom-pair fingerprints are ideal for analyzing large molecules with intricate spatial arrangements, such as proteins, where the relationships between distant atoms are critical to their function.

Together, the integration of ChEMBL's rich dataset and molecular fingerprinting enables powerful computational tools for predicting and designing polymers with specific properties, enhancing the efficiency and precision of polymer discovery.

In the field of polymer discovery, optimization problems with multiple objectives and complex constraints are common. These challenges often arise when balancing conflicting polymer properties, such as strength, flexibility, and thermal stability, while also adhering to specific environmental or performance constraints. Traditionally, mathematical programming techniques like Linear Programming (LP), Mixed-Integer Programming (MIP), and Non-Linear Programming (NLP) have been applied to solve such optimization problems. However, these techniques often encounter limitations when dealing with highly non-linear relationships between polymer properties, large solution spaces, and the diverse nature of polymer formulations.

With the rise of machine learning, particularly neural networks (e.g., CNNs and RNNs), polymer discovery has been transformed. Neural networks excel at learning intricate, non-linear patterns from large datasets, enabling researchers to model and predict complex polymer behaviors that are difficult to capture using traditional optimization methods. By analyzing historical data on polymer formulations, neural networks can identify patterns that link molecular structures with desired properties, such as elasticity or biodegradability. However, while neural networks are powerful for generating potential solutions, they do not always guarantee optimal or feasible results, particularly when complex physical or chemical constraints must be met.

To refine and validate neural network-generated solutions, traditional optimization techniques are still needed. For example, Linear Programming (LP) can be applied to optimize polymer production processes where relationships between variables remain linear, such as minimizing the cost of raw materials or energy usage while maintaining product quality. LP's efficiency, using algorithms like the Simplex method or Interior-Point Methods, makes it a valuable tool in situations where linear constraints dominate.

For more complex decision-making scenarios, Mixed-Integer Programming (MIP) is used when discrete decisions are required, such as determining the optimal number of polymer batches or manufacturing units. MIP can handle these integer constraints and is often used in supply chain optimization and production planning in polymer manufacturing. However, the presence of integer variables makes MIP problems more computationally intensive.

In many cases, Non-Linear Programming (NLP) is needed to model polymer discovery problems that involve non-linear relationships between molecular structure and properties, such as chemical reactivity or stress-strain behavior. NLP is particularly suited for predicting non-linear effects in polymers, but it is more challenging due to the potential for multiple local optima. Techniques like Gradient Descent or Sequential Quadratic Programming (SQP) are often used to navigate the complex landscape of polymer properties, although they may not always guarantee a global optimum.

Physical Programming offers a unique multi-objective optimization approach, particularly useful in polymer discovery where conflicting objectives, like optimizing mechanical strength while minimizing environmental impact, must be balanced. Unlike traditional methods that rely on assigning weights to each objective, physical programming allows decision-makers to specify ranges of desirability for each polymer property (e.g., highly desirable thermal stability, acceptable flexibility, undesirable brittleness). This preference-based approach is especially valuable in polymer development, where trade-offs between different objectives—such as performance, cost, and sustainability—can be difficult to quantify. By translating qualitative preferences into quantitative expressions, physical programming helps guide the optimization process to identify polymer formulations that meet multiple performance criteria.

In summary, combining the predictive power of machine learning models like neural networks with optimization techniques such as LP, MIP, NLP, and Physical Programming allows for a more robust approach to polymer discovery. Machine learning provides the ability to explore vast chemical spaces and predict non-linear relationships, while traditional optimization techniques ensure that solutions are feasible, optimal, and aligned with the specific constraints of polymer production and application. This hybrid approach is accelerating the pace of innovation in the polymer industry, enabling the discovery of materials with tailored properties for specific applications.

Neural networks like RNNs, LSTMs, and CNNs provide innovative and powerful tools for polymer discovery. They enhance the ability to model and predict complex polymer behaviors, leading to more efficient and effective exploration of chemical space and the development of materials with tailored properties. However, challenges such as data availability and model interpretability must be addressed to fully unlock their potential in this domain.

### SUMMARY OF THE INVENTION

The invention combines Convolutional Neural Networks (CNNs) with Physical Programming (PP) to predict and optimize polymer properties using data from the ChEMBL database, creating a robust method for discovering new polymers with tailored characteristics.

The process begins with the data preparation phase, where molecular data from ChEMBL, including descriptors such as molecular weight, A log P, HBA, and HBD, is uploaded. To ensure high-quality inputs, rows containing zero values in key columns are removed. After this cleaning step, molecular structures are transformed into molecular fingerprints, which are binary representations that highlight the relevant structural features of each molecule.

Following data preparation, the next phase involves CNN model training. The CNN uses molecular fingerprints as high-dimensional vectors, and its architecture is composed of convolutional layers to extract local features, pooling layers to reduce the dimensionality, and fully connected layers to classify the molecules based on specific properties, such as solubility or bioactivity. The training process optimizes a loss function by comparing the predicted polymer properties to their true values, ultimately saving the trained model for further use.

Once the CNN has been trained, it is integrated with Physical Programming (PP) to refine the predictions and ensure they align with real-world constraints. In this step, Desirability Functions are introduced to define the ideal range for polymer properties, such as molecular weight and mechanical strength, steering the CNN outputs toward these targets. At the same time, Penalty Functions are applied to impose constraints, penalizing predictions that fall outside the desired range. This ensures the predictions are not only accurate but also practical for real-world applications.

The final step produces the optimized output, where a combined loss function-merging the CNN's prediction errors with physical constraints-ensures that the final predictions meet the necessary real-world requirements. The output includes optimized polymer candidates, each with a target score that reflects how well the predicted properties adhere to the defined criteria, such as thermal stability or mechanical strength. These optimized polymers are ready for further testing or practical use in various applications.

### DETAILED DESCRIPTION OF THE INVENTION

The invention integrates Convolutional Neural Networks (CNNs) with Physical Programming (PP) to optimize the discovery of new polymers with desired properties. This innovative method leverages data from the ChEMBL database and applies machine learning in combination with real-world constraints to generate polymers that meet specific performance criteria. It follows a systematic approach involving data preparation, machine learning, and optimization to predict and refine polymer properties, leading to the development of high-performance polymers.

The invention addresses key polymer properties essential for various applications, such as drug delivery, material science, and industrial processes. These properties include molecular descriptors such as molecular weight, A log P (logarithm of the partition coefficient between n-octanol and water), HBA (hydrogen bond acceptors), and HBD (hydrogen bond donors). These factors serve as objectives in the multi-objective optimization function, allowing for the discovery of polymers with tailored properties.

The process begins building and compiling a 1D CNN model. The function build_model(input_shape) creates and compiles a 1D CNN model for regression tasks. The model begins with Conv1D layers that process the 1D sequential data using a kernel size of 3 and ReLU activation, which helps capture local patterns within the molecular data. Next, MaxPooling1D layers are applied to downsample the data, reducing its dimensionality and computational load. The output from the convolutional layers is then flattened, and a fully connected Dense layer with 128 units and ReLU activation is added to learn complex relationships in the data. A final Dense layer with a single unit and a linear activation function is used for predicting continuous target values, making it suitable for regression tasks. The model is compiled using the Adam optimizer and mean_squared_error as the loss function, which is ideal for regression problems. Additionally, Mean Absolute Error (MAE) is used as a performance metric to evaluate the model's accuracy.

The next step is to load preprocessed molecular data from a database such as an Excel file using the pandas library. Key molecular descriptors such as Molecular Weight, A log P, and Bioactivities are extracted for analysis. To ensure the dataset's quality, the code filters out rows that contain missing (NaN) or zero values in crucial columns, particularly the molecular descriptors. This data cleaning step is essential to maintain accuracy during model training. Next, feature columns are created from the available descriptors, excluding the target variable (like Bioactivities). The target columns include various properties such as Molecular Weight, HBA, HBD, and others, which are used for prediction and further analysis.

The feature data is normalized using StandardScaler, which adjusts the data so that each feature has a mean of 0 and a standard deviation of 1. This normalization is a crucial step for ensuring efficient and effective training of neural networks. After normalization, the data is reshaped into a 3D format, with dimensions representing the number of samples, features, and one channel. This reshaping aligns the data with the input structure required for the 1D CNN model, allowing it to process the data correctly.

The next step is to iterate over each target property, such as Molecular Weight and A log P, training a separate CNN model for each one. For each target property, the data is first split into training and testing sets using train_test_split. A CNN model is then built for the specific target column and trained over 50 epochs with a batch size of 32. Once the training is complete, the trained model is saved as an .h5 file in the designated directory. Each file is named according to the respective target property, such as chembl_cnn_model_for_Molecular_Weight.h5, ensuring organized storage of all models.

Next, the molecular fingerprints are fed into a Convolutional Neural Network (CNN) for training. The CNN architecture is designed to capture critical molecular features using various layers: the input layer receives the molecular fingerprints as high-dimensional vectors, while convolutional layers extract local features corresponding to specific chemical properties such as solubility, mechanical strength, or bioactivity. For model training, the data is first split into training and testing sets using train_test_split for each target property. The CNN model is built for the respective target column and trained over 50 epochs with a batch size of 32. After the training is complete, the model is saved as training files such as an .h5 file in the specified directory, with the file name corresponding to the target property (e.g., chembl_cnn_model_for_Molecular_Weight.h5). Each model is then evaluated using the Mean Absolute Error (MAE) on the test set. Finally, predictions for each target property are generated on the test data, and the script prints both the predictions and actual values for the first few samples to allow for inspection and comparison.

Pooling layers reduce the dimensionality of the data, retaining the most important features while reducing computational load. Fully connected layers aggregate the learned features and classify the molecules based on desired properties, such as solubility or flexibility. The CNN is trained to predict polymer properties using historical data, with a loss function minimizing the difference between the predicted and actual polymer properties. The trained model is saved for future use and integration.

After CNN training, the model is integrated with a Physical Programming (PP) framework to further refine the predictions. This step ensures that the predicted polymer properties meet real-world constraints and desirability criteria, addressing the limitations of purely data-driven models. This process integrates reward and penalty functions with machine learning predictions from pre-trained models saved from the CNN training.

The reward function calculates how close the predicted value is to the center of the desired range for a property. The closer the prediction is to the center, the higher the reward (scaled between 0 and 100).

The equation for the reward function is:

$$Reward ( x ) = max ( 0 , ( 1 - \frac{\vert x - center \vert}{range} ) \times 100 )$$

Where:
x: is the predicted value.

$$center = \frac{desired\_range [ 0 ] - desired\_range [ 1 ]}{2} ,$$

which is the midpoint of the desired range.
range=desired_range[1]−desired_range[0], which is the width of the desired range.
   This equation rewards predictions that are closer to the center of the range, with a reward of 100 for predictions exactly at the center.

The penalty function is applied when the prediction falls outside the desired range. It imposes a quadratic penalty based on how far the prediction is from the acceptable range. The equation for the penalty function is:

$$Penalty ( x ) = \{ \begin{matrix}0 & if desired\_range [ 0 ] \leq x \leq desired\_range [ 1 ] \\ {( desired\_range [ 0 ] - x )}^{2} & if x < desired\_range [ 0 ] \\ {( x - desired\_range [ 1 ] )}^{2} & if x > desired\_range [ 1 ]\end{matrix}$$

Where:
x is the predicted value.
desired_range[0] is the lower bound of the desired range,
desired_range[1] is the upper bound of the desired range.

The total score for the predictions is calculated as the balance between the weighted sum of rewards and the weighted sum of penalties for each property. This allows a balance between encouraging predictions close to the desired ranges (rewards) and penalizing large deviations (penalties).

The equation for the total score is:

$$Total Score = {\sum }_{properties} ( {w}_{prop} \times Reward ( {x}_{prop} ) - {w}_{prop} \times Penalty ( {x}_{prop} ) )$$

Where:
xₚᵣₒₚ is the predicted value for a property.
wₚᵣₒₚ is the weight assigned to each property (optional, depending on whether weights are used).
Reward(xₚᵣₒₚ) is the reward function applied to the predicted value for that property.
Penalty(xₚᵣₒₚ) is the penalty function applied to the predicted value for that property.

If no weights are used, wₚᵣₒₚ is set to 1 for all properties.

If weights are applied to prioritize certain properties over others, the weights are normalized so that the sum of all weights equals 1. The normalization equation is:

$${w}_{prop}^{′} = \frac{{w}_{prop}}{{\sum }_{properties} {w}_{prop}}$$

Where:

$${w}_{prop}^{′}$$

is the original weight assigned to the property.
wₚᵣₒₚ is the original weight assigned to the property.

The sum of the normalized weights is always 1:

$${\sum }_{properties} {w}_{prop}^{′} = 1$$

These equations provide a framework for balancing rewards and penalties to optimize the predictions from the CNN model based on both desirable properties and real-world constraints.

### Advantages of the Invention

The Neural Optimization Platform for Polymer Discovery offers several significant advantages, particularly in tackling complex, multi-objective optimization problems that arise during the polymer discovery process. By combining neural networks such as Convolutional Neural Networks (CNNs) with Physical Programming (PP), this invention leverages the strengths of both machine learning and traditional mathematical optimization techniques.

One of the key advantages is the ability to model non-linear relationships in polymer properties using neural networks. Polymer discovery often involves intricate interactions between molecular structure and properties, such as mechanical strength, flexibility, and thermal stability. Neural networks excel at identifying patterns and generating predictions based on large datasets, which significantly reduces the need for extensive experimental trials. This is particularly beneficial in fields like drug delivery, material science, and industrial processes, where polymers with specific, tailored properties are required.

Another advantage is the integration of Physical Programming, which refines the neural network's predictions by ensuring they meet real-world constraints. The use of Desirability Functions helps guide the predictions toward optimal ranges for polymer properties like molecular weight or mechanical strength, while Penalty Functions impose constraints to avoid impractical outcomes. This combination ensures that the predictions generated are not only accurate but also feasible for practical applications, a limitation that pure machine learning approaches often encounter.

Additionally, the invention supports the use of data from the ChEMBL database, which provides detailed bioactivity data and molecular descriptors. By transforming this molecular data into molecular fingerprints, the CNN models can effectively capture structural information that is crucial for polymer property prediction. This integration of cheminformatics data with machine learning accelerates the discovery process, allowing for faster and more precise development of high-performance polymers.

Finally, the use of traditional optimization techniques, such as Linear Programming (LP), Mixed-Integer Programming (MIP), and Non-Linear Programming (NLP), adds robustness to the overall process. These methods are particularly useful when refining neural network-generated solutions to ensure they adhere to specific production constraints or environmental considerations. For example, LP can be used to minimize the cost of raw materials while maintaining product quality, while NLP is useful in modeling non-linear effects like chemical reactivity in polymers.

### BRIEF DESCRIPTION OF THE DRAWINGS

FIG. 1 illustrates the various methods available for accessing ChEMBL data, offering flexible access through multiple formats and databases: 1, MySQL, 2, CSV (Comma-Separated Values), 3, SQLite, 4, Oracle Database, 5, Excel (XLS), 6, JSON (JavaScript Object Notation), 7, RDF (Resource Description Framework), and 8. the ChEMBL Web Interface. Each of these methods provides access to ChEMBL's comprehensive chemical and bioactivity data, supporting a wide range of applications in research and computational analysis.

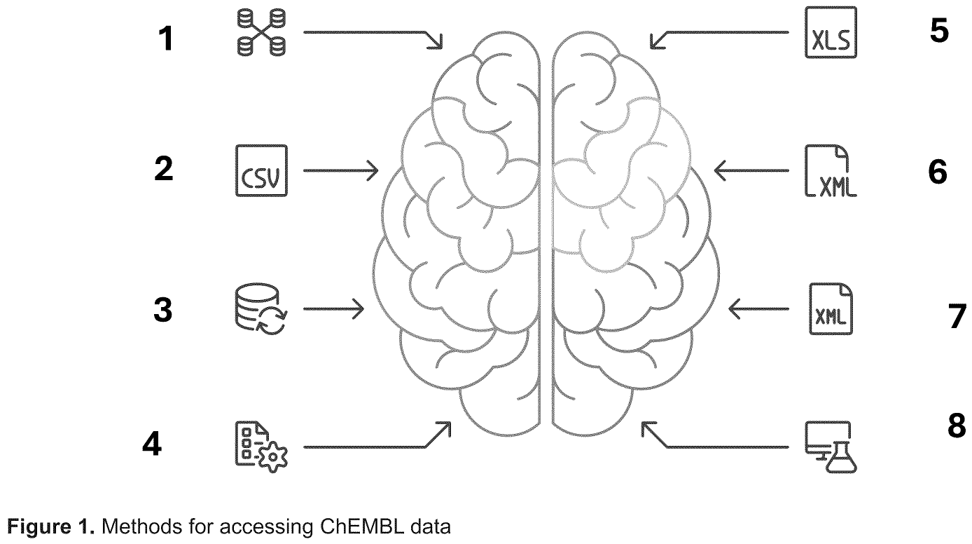

FIG. 2 presents the different types of Molecular Fingerprints and their applications: 1, Substructure Fingerprints is ideal for representing smaller molecules, such as those commonly found in pharmaceuticals. 2, Atom-Pair Fingerprints is optimized for analyzing larger, more complex molecules like peptides.

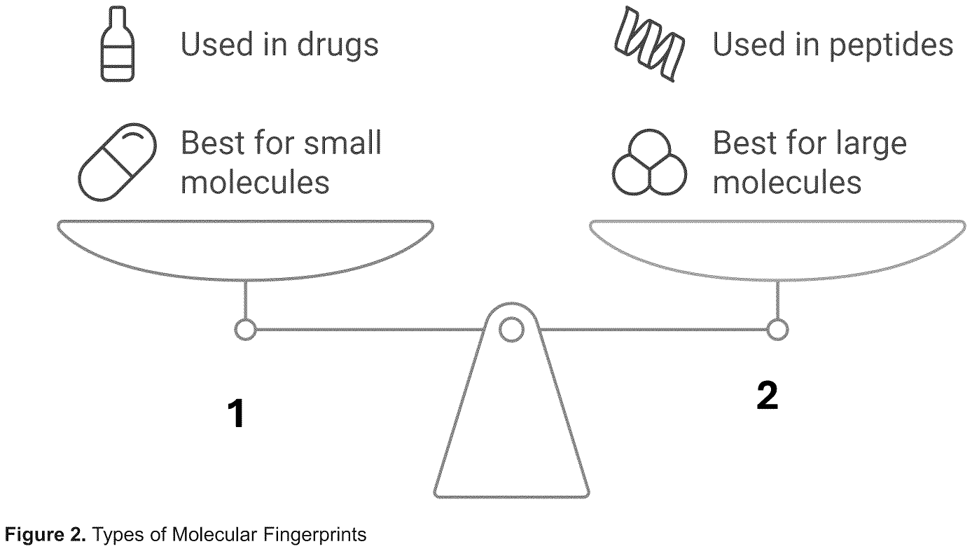

FIG. 3 provides an overview of the 1, ChEMBL Molecular Data categories consisting of 2, Biological Data, including targets and bioactivities, 3, Clinical Data, contains the maximum clinical phase achieved by a compound, 4, Drug-likeness, including properties such as hydrogen bond acceptors (HBA), donors (HBD), and violations of Lipinski's Rule of Five. 5, is Basic Information such as Names, synonyms, and type of compound. 6, is Physical Properties such as Molecular weight, A log P, polar surface area. 7, is Structural Data including Molecular formula, SMILES notation, and InChI Key.

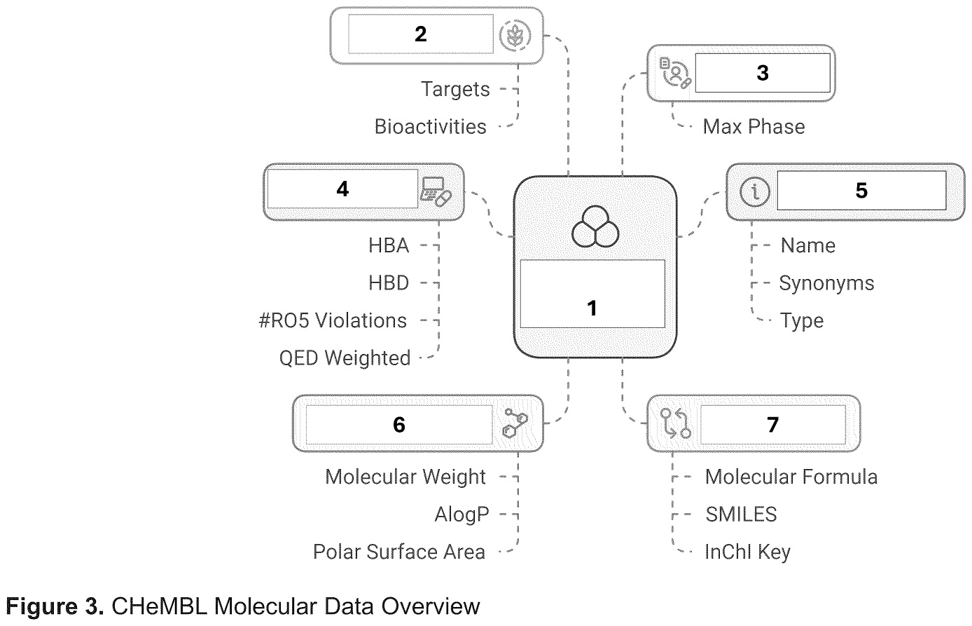

FIG. 4 explains the Data Preparation Process for molecular fingerprints, including, 1, upload from Database. Molecular data is imported from a source like ChEMBL. 2, is data Inspection, data is reviewed for quality and consistency. 3, Prune Zero Values: Rows or columns with zero or missing values are removed. 4, Transform to Fingerprints: Molecular structures are transformed into binary fingerprints, representing the molecular features for input into the model.

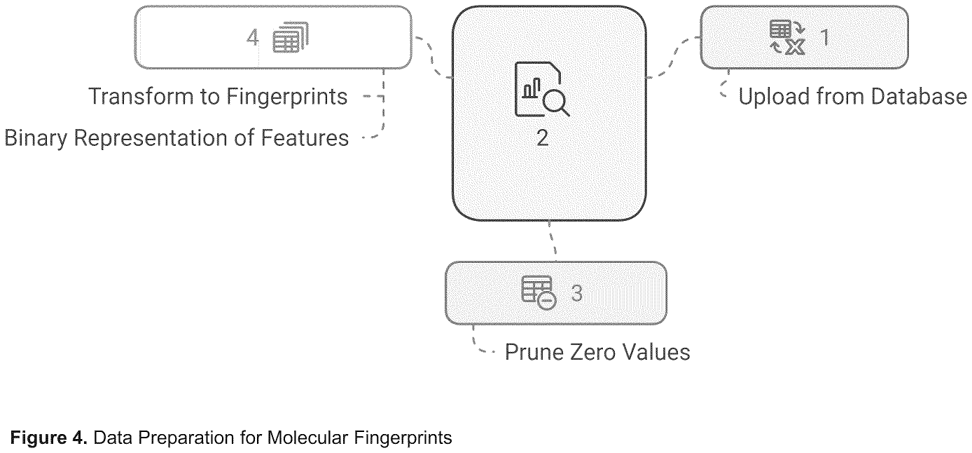

FIG. 5 illustrates the CNN Architecture for Molecular Fingerprint Analysis. 1 is molecular Fingerprint, the input to the CNN, representing chemical structures. 2 is convolution Layer, which extracts local features from molecular fingerprints. 3 is pooling Layer which reduces dimensionality, retaining essential features. 4 is Second Convolution Layer to further extracts complex features. 5 is second pooling layer to further reduce data dimensionality. 7 is flatten layer to convert the data into a format suitable for the fully connected layer. 8 is fully connected layer that aggregates features for classification or prediction. 9 is polymer predictions, based on the molecular fingerprints.

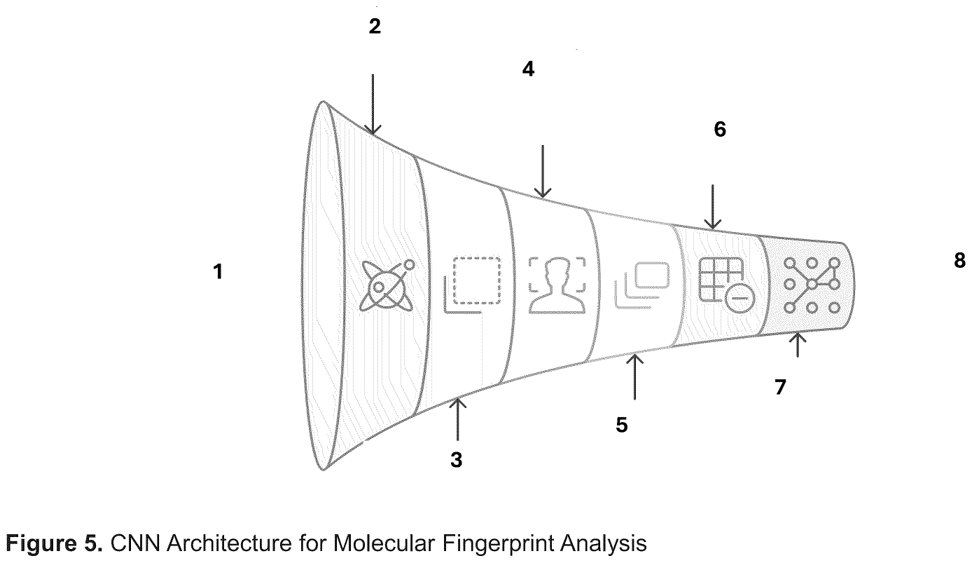

FIG. 6 outlines the Polymer Property Prediction Process. 1 is Data Preparation, here the molecular data is cleaned and prepared. 2 is CNN Model Training. CNN is trained using molecular data to identify patterns. 3 is physical programming integration to optimize CNN predictions under real-world constraints. 4 is optimized output which produces optimized polymer property predictions.

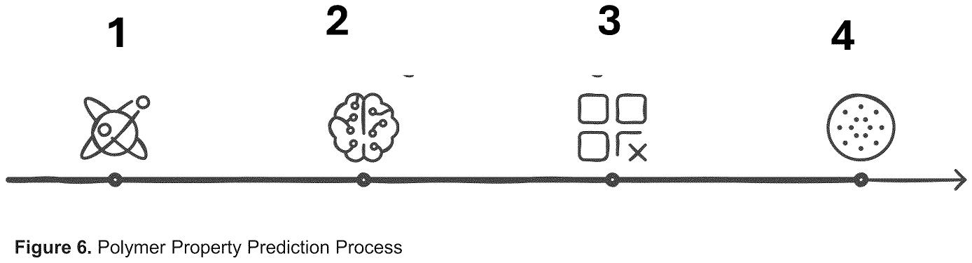

FIG. 7 details the process of Integrating CNN with Physical Programming. 1 is training of the CNN with molecular data. 2 is integrate with PP Framework. Physical programming is integrated to guide CNN predictions. 3 is defining desirability functions. The target range for desired polymer properties is defined. 4 is Penalty Functions. Penalties are applied for deviations from the desired outcome. 5 is Combine Loss Functions. The CNN's loss function is combined with physical programming penalties to optimize predictions, ensuring both accuracy and practicality.

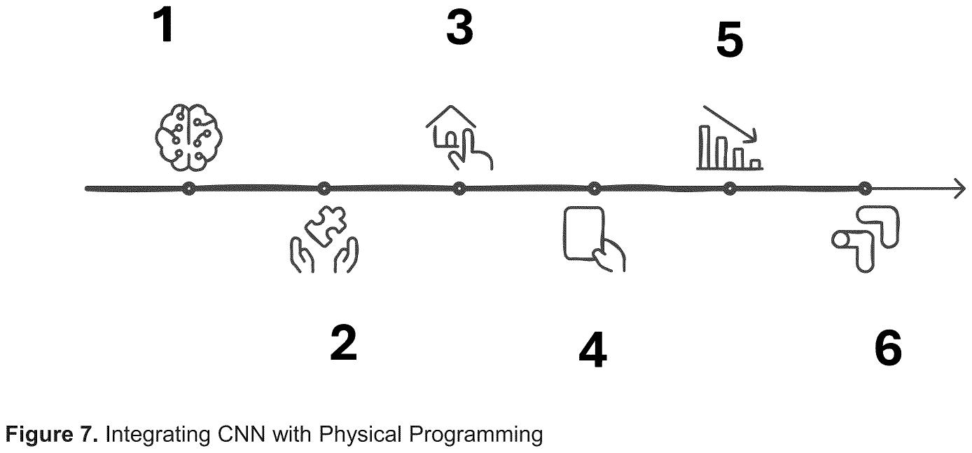

FIG. 8 presents a bar chart illustrating the Total Score for each Target Property.

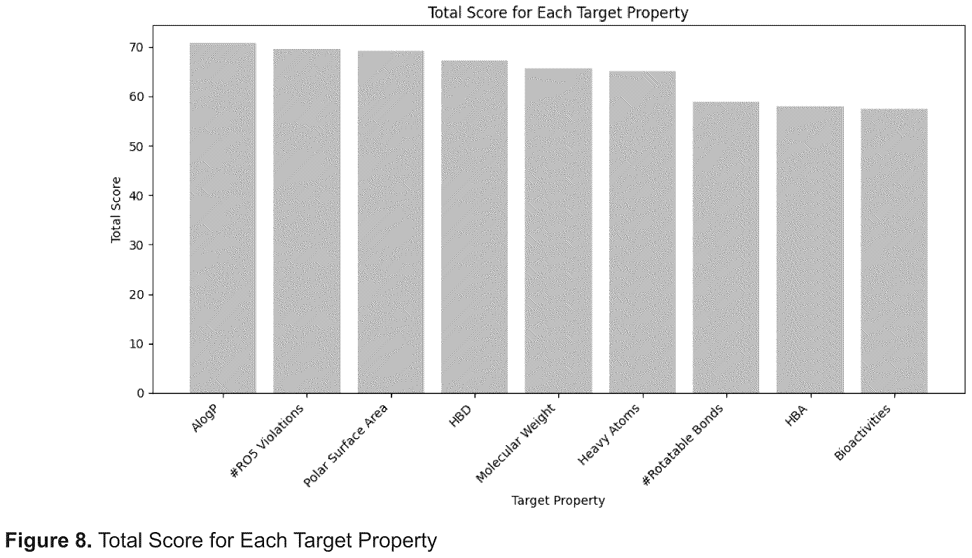

### EXAMPLE. DISCOVERING A BIODEGRADABLE POLYMER FOR DRUG DELIVERY

### Step 1: Data Collection and Preparation

The process begins by collecting molecular data from a source like the ChEMBL database. ChEMBL is a manually curated database of bioactive molecules with drug-like properties. It brings together chemical, bioactivity and genomic data to aid the translation of genomic information into effective new drugs. A protein database consisting of 22,874 compounds was used as the baseline for this example. Initially it was in a csv file, which was subsequently converted to an excel file for ease of use. Table 1 shows the format of the Protein Excel Database. The Excel database detailed molecular and chemical data from ChEMBL, including properties for specific compounds or proteins. Here's a breakdown of key columns:
1. ChEMBL ID: Unique identifier for each compound or protein in the ChEMBL database.
2. Name: The name of the compound or protein.
3. Synonyms: Alternative names or identifiers for the compound, providing flexibility for searches.
4. Type: Classifies the molecule as a protein, small molecule, etc.
5. Max Phase: Refers to the highest clinical trial phase that the compound has reached, indicating how advanced it is in terms of drug development.
6. Molecular Weight: The weight of the molecule, crucial for predicting solubility, permeability, and drug-like properties.
7. Targets: Refers to biological targets, such as proteins, that the compound interacts with.
8. Bioactivities: Represents bioactivity data, measuring the compound's effectiveness in interacting with its target.
9. A log P: A property measuring lipophilicity, which is important for drug absorption and distribution.
10. Polar Surface Area: A measure of the surface area occupied by polar atoms (e.g., oxygen, nitrogen), influencing permeability and solubility.
11. HBA (Hydrogen Bond Acceptors): The number of atoms in the molecule that can accept hydrogen bonds, impacting solubility and interactions with biological targets.
12. HBD (Hydrogen Bond Donors): The number of atoms that can donate hydrogen bonds, similarly affecting solubility and target interactions.
13. #RO5 Violations: Indicates if the compound violates any of Lipinski's “Rule of Five,” which predicts a molecule's drug-likeness.
14. #Rotatable Bonds: The number of bonds that can freely rotate, which can influence molecular flexibility and bioavailability.
15. Structure Type: Classifies whether the molecule is inorganic, organic, or a mixture of both.
16. Heavy Atoms: Number of non-hydrogen atoms, which contributes to molecular complexity and potency.
17. InChI Key/SMILES: These are text representations of the chemical structure of the molecule, useful for cheminformatics and computational analysis.

TABLE 1
Protein Excel Database (Excel file shall be provided)

| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
|---|---|---|---|---|---|---|---|---|---|---|---|---|---|---|---|---|---|---|---|
| [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | | | | | | | | | | | | | | | | [symbol] | [symbol] |
| [symbol] | [symbol] | | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | | | | | | | | | | | | | | | | | [symbol] |
| [symbol] | [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |

[symbol]

indicates data missing or illegible when filed

In this case, the data is preprocessed and filtered to ensure high-quality input for the model. Missing values are removed, and molecular descriptors are transformed into molecular fingerprints, which are compact binary representations that capture structural information about the molecules.

### Step 2: CNN Model Training

Next, the molecular fingerprints are used to train a Convolutional Neural Network (CNN) model.

The provided CNN MODEL POLYMERS code provides the architecture for this process, where the CNN learns to predict various polymer properties, such as degradability and biocompatibility.

The CNN architecture starts by passing the input (molecular fingerprints) through a series of Conv1D and Pooling layers, which identify important local patterns in the molecular structure. After reducing the data's dimensionality using pooling, the network flattens the data and passes it through fully connected layers, which make predictions about the desired properties. The model is trained to minimize errors in predicting these properties, comparing predictions to actual values in the dataset. The model is saved and used for further prediction.

### CNN Model Polymers Code

```
# prompt: mount google drive
from google.colab import drive
drive.mount(‘/content/drive’)
# Step 1: Install and Import Necessary Libraries
!pip install pandas openpyxl tensorflow rdkit-pypi
import os
import pandas as pd
from sklearn.model_selection import train_test_split
from sklearn.preprocessing import StandardScaler
from tensorflow.keras import models, layers
from tensorflow.keras import layers, models
# Function to build the CNN model
def build_model(input_shape):
  model = models.Sequential( )
  # Add layers to the model
  model.add(layers.Conv1D(32, kernel_size=3, activation=‘relu’, input_shape=input_shape))
  model.add(layers.MaxPooling1D(pool_size=2))
  model.add(layers.Conv1D(64, kernel_size=3, activation=‘relu’))
  model.add(layers.MaxPooling1D(pool_size=1)) # Adjust if needed
  model.add(layers.Flatten( ))
  model.add(layers.Dense(128, activation=‘relu’))
  model.add(layers.Dense(1, activation=‘linear’)) # Assuming regression problem
    # Compile the model
  model.compile(optimizer=‘adam’, loss=‘mean_squared_error’, metrics=[‘mae’])
  return model
# Load the data (assuming it is pre-processed)
data_path = ‘/content/drive/MyDrive/Sanjeevakosha/CHEMBL DATA/Protein
Database/Protein.xlsx’ # Replace with actual dataset path
chembl_data = pd.read_excel(data_path)
# Specify the columns to check for non-zero values
columns_to_check = [
  ‘Molecular Weight’, ‘Bioactivities', ‘AlogP’, ‘Polar Surface Area’,
  ‘HBA’, ‘HBD’, ‘#RO5 Violations', ‘#Rotatable Bonds', ‘Heavy Atoms'
]
# Drop rows with NaN or zero in the specified columns
chembl_data_filtered = chembl_data.dropna(subset=columns_to_check)
chembl_data_filtered = chembl_data_filtered[(chembl_data_filtered[columns_to_check] !=
0).all(axis=1)]
# Define feature columns and target columns
feature_columns = [col for col in columns_to_check if col != ‘Bioactivities']
target_columns = [‘Molecular Weight’, ‘Bioactivities', ‘AlogP’, ‘Polar Surface Area’,
  ‘HBA’, ‘HBD’, ‘#RO5 Violations', ‘#Rotatable Bonds', ‘Heavy Atoms']
# Normalize the feature data
scaler = StandardScaler( )
X = scaler.fit_transform(chembl_data_filtered[feature_columns].values)
# Reshape X for CNN (samples, features, 1 channel for 1D CNN)
X = X.reshape((X.shape[0], X.shape[1], 1))
# Split the data into training and testing sets
X_train, X_test, y_train, y_test = train_test_split(X, chembl_data_filtered[‘Bioactivities'],
test_size=0.2, random_state=42)
# Specify the directory where you want to save the models
save_directory = ‘/content/drive/MyDrive/Sanjeevakosha/CHEMBL DATA/Protein Database’ #
Replace with actual path
# Ensure the directory exists, create if it doesn't
if not os.path.exists(save_directory):
  os.makedirs(save_directory)
# Train and save models for each target column
for target_column in target_columns:
  print(f“Training model for target: {target_column}”)
    # Prepare target vector (y) for the current target
  y = chembl_data_filtered[target_column].values
  X_train, X_test, y_train, y_test = train_test_split(X, y, test_size=0.2, random_state=42)
    # Build the model
  model = build_model(input_shape=(X_train.shape[1], 1))
# Train the model
  history = model.fit(X_train, y_train, epochs=50, batch_size=32, validation_data=(X_test,
y_test))
    # Define the filename and save the model
  model_filename = os.path.join(save_directory,
f‘chembl_cnn_model_for_{target_column}.h5’)
  model.save(model_filename)
  print(f“Model saved as {model_filename}”)
    # Evaluate the model on the test set
  loss, mae = model.evaluate(X_test, y_test)
  print(f“Test MAE for {target_column}: {mae}”)
  # Predict on test data
  predictions = model.predict(X_test)
  print(f“Sample Predictions for {target_column}: {predictions[:5].flatten( )}”)
  print(f“Sample True Values for {target_column}: {y_test[:5]}”)
The following training files are created:
chembl_cnn_model_for_#RO5 Violations.h5
chembl_cnn_model_for_#Rotable Bonds.h5
chembl_cnn_model_for_{——}AlogP.h5
chembl_cnn_model_for_{——}Bioactivities.h5
chembl_cnn_model_for_HBA.h5
chembl_cnn_model_for_Heavy Atoms.h5
chembl_cnn_model_for_Molecular Weight.h5
chembl_cnn_model_for_Polar Surface Area.h5
```

Step 3: Prediction and Refinement with Physical Programming

Once CNN has been trained, the Physical Programming (PP) phase refines the predictions. This step is critical because neural networks may predict potential solutions that don't fully adhere to real-world constraints or specific performance targets.

For example, if we need a polymer with an optimal biodegradation rate, CNN might predict polymers that are either too slow or too fast in degrading. The PPBACKUP code shows how the reward function encourages predictions within a desired range, and the penalty function discourages those that fall outside of it.

For our example, the desired range for biodegradability might be 3-6 months, with penalties applied to predictions outside of this range. Likewise, the desired range for molecular weight and drug release rate is defined, and penalties/rewards are applied to optimize these properties. The combined loss function balances both CNN prediction accuracy and adherence to physical constraints.

The desired ranges for each property are specified as constraints that the prediction should ideally fall within. For example:
Molecular Weight: [300, 500]
Bioactivities: [0.1, 0.2]
Polar Surface Area: [60, 140]
HBA (Hydrogen Bond Acceptors): [5, 10]
HBD (Hydrogen Bond Donors): [1, 5]
#RO5 Violations: [0, 1]
#Rotatable Bonds: [0, 5]
Heavy Atoms: [20, 50]
A log P: [2, 5]

One can adjust these ranges based on the specific goals of one's polymer discovery project or optimization needs.

### Physical Programming Code

Reward scales from 0 to 100

### Step 4: Optimized Polymer Discovery

Once the refinement process is complete, the platform generates a set of optimized polymer candidates. Each candidate is evaluated based on how well it meets the desired properties for drug delivery, such as:
Target biodegradability
Controlled drug release rate
Biocompatibility
Molecular weight appropriate for the drug being delivered

The platform assigns a target score to each polymer, reflecting how well the candidate adheres to predefined criteria. Polymers with the highest scores are selected for further testing or practical use. In the context of drug discovery using a protein database, the target score approach can be applied similarly to polymer discovery by evaluating molecular properties that are crucial for drug efficacy. The target score reflects how well a candidate drug molecule aligns with predefined physicochemical properties such as molecular weight, A log P, bioactivities, polar surface area, and other drug-likeness indicators. In this scenario, protein-ligand interactions are assessed, and the model uses the protein database to compare molecular properties of drug candidates to desired criteria, optimizing the selection of viable drug candidates.

This approach not only helps streamline the drug discovery process but ensures that the identified molecules conform to the biological and chemical constraints necessary for efficacy, bioavailability, and safety, often using machine learning techniques and physical programming to refine the results based on these multi-objective criteria.

TABLE 2
Physical Programming Results (Excel file shall be provided)

| | Molecular | | Polar Surface | | | #RO5 | #Rotatable | Heavy | | Total |
|---|---|---|---|---|---|---|---|---|---|---|
| Target | Weight | Bioactivities | Area | HBA | HBD | Violations | Bonds | Atoms | AlogP | Score |
| Molecular Weight | 449.47 | 0.129 | 92.652 | 7.805 | 2.888 | 0.913 | 3 | 38 | 4.605 | 80.4 |
| HBD | 436.25 | 0.171 | 100.149 | 5.432 | 3.837 | 0.496 | 5 | 48 | 2.101 | 77.1 |
| #Rotatable Bonds | 444.34 | 0.124 | 92.577 | 9.996 | 2.348 | 1.188 | 6 | 51 | 3.408 | 72.5 |
| #RO5 Violations | 301.64 | 0.139 | 139.923 | 8.532 | 1.570 | 1.504 | 2 | 50 | 3.836 | 70.3 |
| AlogP | 314.05 | 0.087 | [symbol] 7.441 | 10.445 | 3.242 | 0.253 | 3 | 20 | 4.765 | 67.8 |
| HSA | 350.39 | 0.123 | 66.325 | 6.921 | 0.562 | 1.458 | 2 | 35 | 4.381 | 66.2 |
| Polar Surface Area | 335.10 | 0.192 | 81.493 | 9.788 | 1.645 | 1.507 | 2 | 29 | 2.318 | 65. [symbol] |
| Bioactivities | 383.52 | 0.068 | [symbol] 42.717 | 7.645 | 1.489 | 1.543 | 4 | 41 | 2.797 | 61.3 |
| Heavy Atoms | 448.70 | 0.158 | [symbol] 46.252 | 10.714 | 0.769 | 1.945 | 5 | 34 | 2.106 | [symbol] 1.8 |

[symbol]

indicates data missing or illegible when filed

The Neural Optimization Platform for Polymer Discovery offers a wide range of applications across various industries by combining neural networks with optimization techniques to accelerate material discovery and enhance the precision of polymer design. This platform is particularly useful for creating polymers with tailored properties suited for specific needs.

In drug delivery systems, the platform helps design polymers with optimized biocompatibility, degradability, and drug release profiles, ensuring efficient drug delivery with minimal side effects. For biodegradable polymers, the platform aids in developing materials used in packaging, medical implants, and other environmentally friendly applications by optimizing factors such as degradation time and mechanical strength. In material science, it can fine-tune properties like thermal stability, mechanical strength, and adhesion for use in coatings, adhesives, and composites, including self-healing polymers for high-performance industries like aerospace and construction.

The platform also plays a role in electronics, where it designs conductive polymers with the right balance of electrical conductivity and flexibility for applications like flexible electronics, organic photovoltaics, and wearables. For sustainable polymers, the platform supports the creation of materials from renewable sources, optimizing biodegradability and energy-efficient production processes, while in the medical devices sector, it helps develop polymers for implants, prosthetics, and tissue scaffolds that meet biocompatibility and regulatory standards.

In 3D printing and additive manufacturing, the platform optimizes polymers for different printing techniques by refining properties like melt viscosity and layer adhesion, while in coatings and films, it enhances attributes like UV resistance and scratch resistance for use in industries such as automotive and construction. The platform can also assist in creating automotive and aerospace materials, where lightweight polymers with high strength-to-weight ratios are crucial for reducing fuel consumption and improving performance.

For energy storage systems, the platform aids in developing advanced polymers for batteries and supercapacitors by optimizing properties like ionic conductivity and chemical resistance. In water treatment and environmental applications, it helps design polymers for filtration membranes and pollution control efforts, improving sustainability and cleanup efficiency.

In space exploration, particularly for Martian and Lunar environments, the platform is essential for developing polymers that can withstand extreme conditions like high radiation, low atmospheric pressure, and temperature fluctuations. It can optimize polymers for radiation shielding, thermal insulation, and mechanical strength, as well as create self-healing materials for habitat construction. The platform also supports life support systems, optimizing polymers for air and water filtration and oxygen production in reduced gravity conditions. In space suits and extravehicular activity (EVA) equipment, the platform designs polymers that balance flexibility, durability, and radiation resistance while optimizing materials for dust repellency during Martian exploration.

Finally, for In-Situ Resource Utilization (ISRU) on Mars and the Moon, the platform can develop polymers from local resources like regolith for building materials, habitats, and tools, reducing the need for transporting materials from Earth. These polymers can also be used for additive manufacturing, enabling rapid construction of habitats and infrastructure using 3D printing.

1: A computerized method for discovering polymers, comprising:
Extracting molecular data from a chemical database using a computer;
Transforming the molecular data into molecular fingerprints using a computer processor;
Training a Convolutional Neural Network (CNN) model on said fingerprints to predict polymer properties;
Refining said predictions using Physical Programming (PP) executed by the computer, where desirability and penalty functions guide optimization;
Outputting optimized polymer candidates via the computer that meet predefined property constraints.

2: The computerized method of claim 1, wherein the chemical database is the ChEMBL database, and the molecular data includes descriptors such as molecular weight, A log P, and hydrogen bond acceptors/donors, retrieved and processed by the computer.

3: The computerized method of claim 1, wherein the CNN model comprises:
Convolutional layers for extracting molecular features;
Pooling layers for dimensionality reduction;
Fully connected layers for property prediction, implemented through a computer processor.

4: The computerized method of claim 1, wherein the Physical Programming optimization includes:
Desirability functions defining optimal polymer property ranges, computed by the processor;
Penalty functions applied to penalize predictions falling outside said ranges;
A combined loss function balancing CNN prediction accuracy and real-world constraints, executed by the computer.

5: The computerized method of claim 1, wherein the optimized polymer properties include biodegradability, mechanical strength, thermal stability, and solubility, determined through machine learning processes running on the computer.

6: The computerized method of claim 1, wherein the output includes a target score, generated by the computer, reflecting how closely the polymer predictions align with predefined property criteria.

7: A computerized system for polymer discovery, comprising:
A CNN model trained on molecular fingerprint data for predicting polymer properties;
A Physical Programming module integrated into the system to refine CNN outputs and ensure adherence to predefined property constraints.
